(19) United States
(12) Patent Application Publication
(10) Pub. No.: US 2013/0079534 A1
(43) Pub. Date: Mar. 28, 2013

(54) METHOD FOR PRODUCING OLEFIN OXIDE

(71) Applicant: Hideo Kanazawa, Toyonaka-shi (JP)

(72) Inventor: Hideo Kanazawa, Toyonaka-shi (JP)

(73) Assignee: SUMITOMO CHEMICAL COMPANY, LIMITED, Chuo-ku, Tokyo (JP)

(21) Appl. No.: 13/700,556

(22) PCT Filed: May 19, 2011
(86) PCT No.: PCT/JP2011/062040

§ 371 Date: Nov. 28, 2012

(30) Foreign Application Priority Data
May 31, 2010 (JP) 2010-124101

(51) Int. Cl. C07D 301/12

(52) U.S. Cl. CPC C07D 301/12
USPC 549/523

(57) ABSTRACT

According to a conventional method for producing an olefin oxide, hydrogen peroxide and an olefin oxide as a product are obtained in the state of a mixture, and in order to decrease the content of hydrogen peroxide in the mixture, it is necessary to distill the mixture to separate hydrogen peroxide from the olefin oxide. The present invention provides a method for producing an olefin oxide including a reaction step of reacting hydrogen peroxide with an olefin in the presence of a solvent and a titanium silicate catalyst; and a step of mixing a reducing agent containing at least one selected from the group consisting of a sulfide and hydrazine with the reaction solution obtained in the reaction step.

### TECHNICAL FIELD

This application is a National Stage of International Application No. PCT/JP2011/062040, filed on May 19, 2011, which claims priority from Japanese Patent Application No. 2010-124101, filed on May 31, 2010, the contents of all of which are incorporated herein by reference in their entirety.

The present invention relates to a method for producing an olefin oxide, and the like.

### BACKGROUND ART

As a method for producing propylene oxide, which is one kind of olefin oxides, for example, Patent Document 1 describes a method of supplying propylene and hydrogen peroxide into a reaction zone in which an epoxidation catalyst is held; obtaining a mixture of unreacted propylene and hydrogen peroxide, and propylene oxide as a product, in the reaction zone; then supplying the mixture to a distillation zone; and separating the mixture into an overhead fraction containing propylene and propylene oxide, and a bottom fraction containing hydrogen peroxide.

### PRIOR ART DOCUMENT

### Patent Document

[Patent Document 1] JP-A-2004-525073 ([Claim 1] and [Examples])

### SUMMARY OF THE INVENTION

That is, the present invention provides the following:

<1> A method for producing an olefin oxide, including:

a reaction step of reacting hydrogen peroxide with an olefin in the presence of a solvent and a titanium silicate catalyst; and

a step of mixing a reducing agent containing at least one selected from the group consisting of a sulfide and hydrazine with the reaction solution obtained in the reaction step;

<2> The method according to <1>, wherein the reducing agent is sodium sulfide;
<3> The method according to <1>, wherein the reducing agent is a hydrazine hydrate or an aqueous solution of hydrazine;
<4> The method according to any one of <1> to <3>, wherein the olefin is propylene, and the olefin oxide is propylene oxide;
<5> The method according to any one of <1> to <4>, wherein the solvent is a mixed solvent of acetonitrile and water;
<6> The method according to any one of <1> to <5>, wherein the titanium silicate catalyst is a Ti-MWW precursor having a molar ratio of silicon to nitrogen (an Si/N ratio) of 5 to 20;
<7> A method for producing an olefin oxide, including:

a step of continuously adding hydrogen peroxide and an olefin to a reactor in which a solvent and a titanium silicate catalyst are contained, performing reaction in the reactor, and continuously supplying the obtained reaction solution to a decomposition tank; and

a step of continuously supplying the reaction solution obtained in the above-mentioned step, and a reducing agent containing at least one selected from the group consisting of a sulfide and hydrazine to a decomposition tank to continuously obtain a solution containing an olefin oxide;

<8> A method for decreasing an amount of hydrogen peroxide in a solution containing an olefin oxide, including:

a step of mixing a solution containing hydrogen peroxide and an olefin oxide with a reducing agent containing at least one selected from the group consisting of a sulfide and hydrazine to decompose hydrogen peroxide.

### Effect of the Invention

According to the production method of the present invention, an olefin oxide having a decreased content of hydrogen peroxide can be provided without distillation for separating the olefin oxide from hydrogen peroxide.

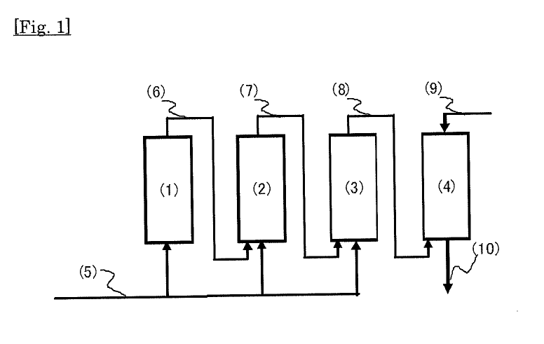

### BRIEF DESCRIPTION OF THE DRAWING

[FIG. 1] One embodiment of an apparatus for producing an olefin oxide.

### MODES FOR CARRYING OUT THE INVENTION

The present invention includes a reaction step of reacting hydrogen peroxide with an olefin in the presence of a solvent and a titanium silicate catalyst.

The olefin in the present invention refers to a compound having a carbon-carbon double bond in its molecule, in which a hydrocarbyl group having 1 to 12 carbon atoms, which may have a substituent, or a hydrogen atom is bonded to the carbon-carbon double bond.

Examples of the substituent for the hydrocarbyl group include a hydroxyl group, a halogen atom, a carbonyl group, an alkoxycarbonyl group, a cyano group, and a nitro group. Examples of the hydrocarbyl group include a saturated hydrocarbyl group, and examples of the saturated hydrocarbyl group include an alkyl group.

Specific examples of the olefin include an alkene having 2 to 10 carbon atoms, and a cycloalkene having 4 to 10 carbon atoms.

Examples of the alkene having 2 to 10 carbon atoms include ethylene, propylene, butene, pentene, hexene, heptene, octene, nonene, decene, 2-butene, isobutene, 2-pentene, 3-pentene, 2-hexene, 3-hexene, 4-methyl-1-pentene, 2-heptene, 3-heptene, 2-octene, 3-octene, 2-nonene, 3-nonene, 2-decene, and 3-decene.

Examples of the cycloalkene having 4 to 10 carbon atoms include cyclobutene, cyclopentene, cyclohexene, cycloheptene, cyclooctene, cyclononene, and cyclodecene.

A more preferable olefin is propylene.

As propylene, propylene which is produced by, for example, thermal cracking, catalytic cracking of heavy oils, or methanol-catalytic reforming is exemplified. Purified propylene, or crude propylene which has not passed through a purification step may be used as propylene.

As described above, in the present invention, crude propylene may be used as the olefin, but a preferable purity of propylene is, for example, 90% by volume or more, more preferably 95% by volume or more. Examples of the impurity contained in crude propylene include propane, cyclopropane, methyl acetylene, propadiene, butadiene, butanes (n-butane and isobutane), butenes (1-butene and 2-butene), ethylene, ethane, methane, and hydrogen.

The amount of the olefin used in the reaction step can be adjusted according to the kind thereof, the reaction condition or the like, and it is preferably at least 0.01 part by weight, more preferably at least 0.1 part by weight based on 100 parts by weight of the total amount of solvents used in the reaction step.

The olefin used in the present invention may be either in the state of a gas or a liquid. Here, examples of the liquid olefin include a mixed liquid of an organic solvent or a mixed solvent of an organic solvent and water, and an olefin dissolved therein, in addition to a liquid of an olefin alone. Examples of the gaseous olefin include a gaseous olefin, and a mixed gas of a gaseous olefin and another gas component such as a nitrogen gas and a hydrogen gas.

The olefin oxide refers to an oxirane compound in which a carbon-carbon double bond of an olefin is replaced by a oxiranyl group, and examples thereof include oxirane compounds having 2 to 10 carbon atoms such as ethylene oxide (oxirane), propylene oxide (1-methyl oxirane), 1-ethyl oxirane, 1-propyl oxirane, 1-butyl oxirane, 1-pentyl oxirane, 1-hexyl oxirane, 1-heptyl oxirane, 1-octyl oxirane, 1-methyl-2-ethyl oxirane and 1-methyl-2-methyl oxirane. For example, when propylene is used as the olefin, the obtained olefin oxide is propylene oxide.

The titanium silicate catalyst used in the present invention refers to a titanosilicate substantially having four-coordinated Ti, in which the maximum absorption peak of an ultraviolet and visible absorption spectrum in a wavelength range of 200 nm to 400 nm appears in a wavelength range of 210 nm to 230 nm (see, for example, “Chemical Communications” 1026-1027, (2002), FIGS. 2(d) and (e)). The ultraviolet and visible absorption spectrum can be measured by using an ultraviolet and visible spectrophotometer equipped with a diffuse reflector in accordance with a diffuse reflection method.

In the present invention, titanosilicate catalysts having fine pores of not less than 10-membered oxygen ring are preferable, because contact inhibition between starting materials for the reaction and active points in the fine pores tends to be suppressed, or limitation of mass transfer in the fine pores tends to be decreased.

The fine pore herein refers to a pore having an entrance in which a ring structure is formed by an Si—O bond and/or a Ti—O bond. The fine pore may be in the state of a half cup called a side pocket.

The phrase “not less than 10-membered oxygen ring” means that when (a) a cross-section of the narrowest part of the fine pore, or (b) a ring structure at the fine pore entrance is observed, the cross-section or the fine pore entrance has a ring structure composed of an Si—O bond and/or a Ti—O bond having 10 or more oxygen atoms.

The fact that a titanosilicate catalyst has fine pores of not less than 10-membered oxygen ring is generally confirmed by an analysis of an X-ray diffraction pattern, and if the catalyst has a known structure, it can be easily confirmed by comparison with an X-ray diffraction pattern of the known one.

Examples of the preferable titanosilicate catalysts in the present invention include titanosilicates 1 to 7 described below.

### 1. Crystalline Titanosilicate Having Fine Pores of 10-Membered Oxygen Ring:

In the IZA (International Zeolite Association) structure code, TS-1 having the MFI structure (for example, U.S. Pat. No. 4,410,501), TS-2 having the MEL structure (for example, Journal of Catalysis 130, 440-446, (1991)), Ti-ZSM-48 having the MRE structure (for example, Zeolites 15, 164-170, (1995)), Ti-FER having the FER structure (for example, Journal of Materials Chemistry 8, 1685-1686 (1998)), and the like.

### 2. Crystalline Titanosilicate Having Fine Pores of 12-Membered Oxygen Ring:

Ti-Beta having a BEA structure (for example, Journal of Catalysis 199, 41-47, (2001)), Ti-ZSM-12 having an MTW structure (for example, Zeolites 15, 236-242, (1995)), Ti-MOR having an MOR structure (for example, The Journal of Physical Chemistry B 102, 9297-9303, (1998)), Ti-ITQ-7 having an ISV structure (for example, Chemical Communications 761-762, (2000)), Ti-MCM-68 having an MSE structure (for example, Chemical Communications 6224-6226, (2008)), Ti-MWW having an MWW structure (for example, Chemistry Letters 774-775, (2000)), and the like.

### 3. Crystalline Titanosilicate Having Fine Pores of 14-Membered Oxygen Ring:

Ti-UTD-1 having a DON structure (for example, Studies in Surface Science and Catalysis 15, 519-525, (1995)), and the like.

### 4. Layered Titanosilicate Having Fine Pores of 10-Membered Oxygen Ring:

Ti-ITQ-6 (for example, Angewandte Chemie International Edition 39, 1499-1501, (2000)), and the like.

### 5. Layered Titanosilicate Having Fine Pores of 12-Membered Oxygen Ring:

A Ti-MWW precursor (for example, EP-1731515-A1), Ti-YNU-1 (for example, Angewandte Chemie International Edition 43, 236-240, (2004)), Ti-MCM-36 (for example, Catalysis Letters 113, 160-164, (2007)), Ti-MCM-56 (for example, Microporous and Mesoporous Materials 113, 435-444, (2008)), and the like.

### 6. Mesoporous Titanosilicate:

Ti-MCM-41 (for example, Microporous Materials 10, 259-271, (1997)), Ti-MCM-48 (for example, Chemical Communications 145-146, (1996)), Ti-SBA-15 (for example, Chemistry of Materials 14, 1657-1664, (2002)), and the like.

### 7. Silylated Titanosilicate:

compounds obtained by silylating the titanosilicates 1 to 6 described above, such as silylated Ti-MWW.

The layered titanosilicate is a generic name of titanosilicates having a layered structure, such as layered precursors of a crystalline titanosilicate, and a titanosilicate in which spaces between layers in a crystalline titanosilicate are expanded. Whether a titanosilicate has a layered structure or not can be confirmed by an electron microscope or measurement of an X-ray diffraction pattern.

The layered precursor refers to a titanosilicate which forms a crystalline titanosilicate by a treatment such as dehydration condensation. It can be easily determined that a layered titanosilicate has fine pores of not less than 12-membered oxygen ring from the structure of a corresponding crystalline titanosilicate.

The mesoporous titanosilicate is a generic name of titanosilicates having regular mesofine pores. The regular mesopore refers to a structure in which mesopores are regularly and repeatedly arranged.

The mesofine pore refers to a fine pore having a diameter of 2 nm to 10 nm.

The silylated titanosilicate can be obtained by treating the titanosilicates 1 to 4 described above with a silylating agent. Examples of the silylating agent include 1,1,1,3,3,3-hexamethyl disilazane and trimethylchlorosilane (for example, EP-1488853-A1).

A titanosilicate catalyst which has been contacted with hydrogen peroxide is preferable. Hydrogen peroxide is subjected to the contact in a concentration of, for example, 0.0001 to 50% by weight.

As the titanosilicate catalyst, for example, titanosilicates having fine pores of not less than 12-membered oxygen ring are preferable, and such titanosilicates may be crystalline titanosilicates or layered titanosilicates. Examples of the titanosilicate having fine pores of not less than 12-membered oxygen ring include Ti-MWW and Ti-MWW precursors.

The Ti-MWW precursor is a generic name of compounds which provide a crystalline titanosilicate having an MWW (a structure code of IZA (International Zeolite Association)) structure by calcination thereof. A crystalline titanosilicate obtained by calcination of a Ti-MWW precursor is a generic name of compounds in which a part of Si atoms in a tetrasilicate are isomorphously substituted by Ti atoms (see the description in the item “Titanosilicate” of Encyclopedia of Catalyst (Asakura Publishing Co., Ltd.) published on Nov. 1, 2000)). The isomorphous substitution of Si by Ti can be easily confirmed, for example, from the appearance of a peak in a range of 210 nm to 230 nm in an ultraviolet and visible absorption spectrum (measured by using an ultraviolet and visible spectrophotometer (V-7100 manufactured by JASCO Corporation) equipped with a diffuse reflector (Praying Mantis manufactured by HARRICK)).

Examples of the method for producing a Ti-MWW precursor include:

a method in which a layered compound (which is also referred to as an “as-synthesized sample”), which is directly hydrothermally synthesized from a boron compound, a titanium compound, a silicon compound and a structure-directing agent, is brought into contact with an aqueous strong acid solution under reflux conditions, the structure-directing agent is removed, and the molar ratio of silicon to nitrogen (Si/N ratio) is adjusted to 21 or more to synthesize the precursor (see, for example, JP-A-2005-262164);

a method in which Ti-MWW, a structure-directing agent such as piperidine, and water are mixed to obtain a compound, and the resulting compound is hydrothermally treated and then washed with water (Catalysis Today, 117 (2006) 199-205); and

a method in which a mixture containing a structure-directing agent, a boron compound, a silicon compound and water is heated to obtain layered borosilicate, the layered borosilicate is brought into contact with, preferably, an acid, or the like to remove the structure-directing agent, the resulting product is calcined to obtain B-MWW, the resulting B-MWW is treated with an acid, or the like to remove boron, a structure-directing agent, a titanium compound and water are added thereto to obtain a mixture, the obtained mixture is heated to obtain a layered compound, and the layered compound is brought into contact with 6 M nitric acid to remove the structure-directing agent (see, for example, Chemical Communication, 1026-1027, (2002)).

Another method for producing a Ti-MWW precursor is a method in which a titanosilicate having an X-ray diffraction pattern with values described below is brought into contact with a structure-directing agent capable of forming zeolite having an MWW structure to obtain the precursor. X-ray diffraction pattern

(Lattice Spacing d/Å)
12.4±0.8
10.8±0.3
9.0±0.3
6.0±0.3
3.9±0.1
3.4±0.1

These X-ray diffraction patterns can be measured by using a general X-ray diffraction apparatus using copper K-α radiation.

Examples of the titanosilicate having the X-ray diffraction pattern described above include titanosilicates described in JP-A-2005-262164, Ti-YNU-1 (for example, titanosilicates described in Angewandte Chemie International Edition, 43, 236-240, (2004)), crystalline titanosilicates, Ti-MWW which is a crystalline titanosilicate having an MWW structure in the IZA (International Zeolite Association) structure code (for example, titanosilicates described in JP-A-2003-327425), and Ti-MCM-68 which is a crystalline titanosilicate having an MSE structure in the IZA structure code (for example, titanosilicates described in JP-A-2008-50186).

Examples of the structure-directing agent used in the present invention include piperidine, hexamethyleneimine, N,N,N-trimethyl-1-adamantane ammonium salts (for example, N,N,N-trimethyl-1-adamantane ammonium hydroxide, and N,N,N-trimethyl-1-adamantane ammonium iodide), and octyl trimethyl ammonium salts (for example, octyl trimethyl ammonium hydroxide and octyl trimethyl ammonium bromide) (see, for example, Chemistry Letters, 916-917 (2007)). Of these, preferable structure-directing agents are piperidine and hexamethyleneimine. These structure-directing agents may be used alone, or as a mixture of two or more kinds thereof in any ratio.

The structure-directing agent is used in an amount of, for example, 0.001 part by weight to 100 parts by weight, preferably 0.1 part by weight to 10 parts by weight per 1 part by weight of the titanosilicate.

The titanosilicate having the X-ray diffraction pattern with the values described above may be brought into contact with the structure-directing agent capable of forming zeolite having an MWW structure by a method of putting them in a sealed container such as an autoclave and heating them under pressure, or a method of mixing them in a glass flask in the atmosphere by stirring, or without stirring. The temperature is preferably from 0° C. to 250° C., and a particularly preferable temperature range is from 50° C. to 200° C. The contact pressure is, for example, from about 0 to 10 MPa in a gauge pressure. After the contact, the obtained Ti-MWW precursor is usually separated by filtration. If necessary, the precursor is further washed with water or the like, thus resulting in obtaining a Ti-MWW precursor having an Si/N ratio of 5 to 20. The washing may be properly performed while observing the amount of a washing liquid or the pH of a wash filtrate, as occasion demands.

A preferable titanosilicate catalyst in the present invention is Ti-MWW precursors having a molar ratio of silicon to nitrogen (Si/N ratio) of 5 to 20, preferably 8.5 to 8.6. Here, an Si/N ratio of a Ti-MWW precursor can be obtained as follows.

First, a Ti-MWW precursor is molten in an alkali and dissolved in nitric acid, and then the content of Si (silicon) in the Ti-MWW precursor is obtained by an ICP emission spectrometry (contents of Ti (titanium) and B (boron) can also be measured at this time). Separately, the Ti-MWW precursor is subjected to oxygen cycle combustion, and its N (nitrogen) content is measured in accordance with a TCD detection method (Sumigraph NCH-22F (manufactured by Sumika Chemical Analysis Service, Ltd.) was used in Examples of the instant specification). Then the molar ratio of silicon to nitrogen (Si/N ratio) can be obtained from the thus obtained results.

Ti-MWW having a peak in 210 nm to 230 nm of an ultraviolet and visible absorption spectrum can be obtained by calcining the Ti-MWW precursor described above at a temperature of 450 to 600° C.

The thus obtained Ti-MWW has an Si/N ratio of 10 to 20, preferably 10 to 16. Further, the Ti-MWW may be silylated using a silylating agent such as 1,1,1,3,3,3-hexamethyl disilazane.

Examples of the titanosilicate catalyst in the present invention include titanosilicate catalysts having a ratio of a specific surface area (SH₂O) measured by a water vapor adsorption method to a specific surface area (SN₂) measured by a nitrogen adsorption method (SH₂O/SN₂) of, for example, 0.7 to 1.5, preferably 0.8 to 1.3. The specific surface area (SN₂) in accordance with the nitrogen adsorption method is obtained by degassing a sample at 150° C., performing measurement by using, for example, “BELSORP-mini” (manufactured by BEL Japan, Inc.) in accordance with the nitrogen adsorption method, and calculating the value in accordance with a BET method.

The specific surface area (SH₂O) in accordance with the water vapor adsorption method is obtained by degassing a sample at 150° C., performing measurement by using, for example, “BELSORP-aqua 3” (manufactured by BEL Japan, Inc.) at an adsorption temperature of 298 K in accordance with the water vapor adsorption method, and calculating the value in accordance with the BET method.

In the reaction step in the present invention, the amount of the titanosilicate catalyst may be suitably selected according to the kind of the reaction. The lower limit thereof is, for example, 0.01 part by weight, preferably 0.1 part by weight, more preferably 0.5 part by weight; and the upper limit thereof is, for example, 20 parts by weight, preferably 10 parts by weight, more preferably 8 parts by weight, based on 100 parts by weight of the total amount of solvents used in the reaction step.

As hydrogen peroxide used in the reaction step, commercial products may be used, or hydrogen peroxide may be generated from oxygen and hydrogen in the presence of a noble metal catalyst, as described later. Hydrogen peroxide may also be supplied to the reaction step in the state of a solution in a solvent described later, such as water or acetonitrile.

In the reaction step, the concentration of hydrogen peroxide is within a range of, for example, 0.0001% by weight to 100% by weight, preferably 0.001% by weight to 5% by weight. The ratio of hydrogen peroxide to the olefin is within a rang of, for example, olefin:hydrogen peroxide=1000:1 to 1:1000 (a molar ratio).

When hydrogen peroxide is produced from oxygen and hydrogen, a noble metal catalyst is used. Here, examples of the noble metal catalyst include catalysts containing a noble metal such as palladium, platinum, ruthenium, rhodium, iridium, osmium or gold, or an alloy or a mixture thereof. Preferable examples of the noble metal include palladium, platinum, and gold, and a more preferable noble metal is palladium. As palladium, for example, palladium colloid may be used (see, for example, JP-A-2002-294301, Example 1). A palladium compound is a preferable noble metal. When a palladium compound is used as the noble metal catalyst, a metal other than palladium such as platinum, gold, rhodium, iridium or osmium may be used by adding the metal to the palladium compound and mixing them. Examples of the preferable metal other than palladium include gold and platinum.

Examples of the palladium compound include tetravalent palladium compounds such as sodium hexachloropalladate (IV) tetrahydrate and potassium hexachloropalladate (IV); and divalent palladium compounds such as palladium (II) chloride, palladium (II) bromide, palladium (II) acetate, palladium (II) acetylacetonate, dichlorobis(benzonitrile)palladium (II), dichlorobis(acetonitrile)palladium (II), dichloro(bis(diphenylphosphino)ethane)palladium (II), dichlorobis(triphenylphosphine)palladium (II), dichlorotetraamminepalladium (II), dibromotetraamminepalladium (II), dichloro(cycloocta-1,5-diene)palladium (II), and palladium (II) trifluoroacetate.

It is preferable to use the noble metal in the state in which it is supported on a carrier, preferably the titanium silicate catalyst described above. The noble metal may be used in the state in which it is supported on as an oxide such as silica, alumina, titania, zirconia or niobia; a hydrate of niobic acid, zirconic acid, tungstic acid or titanic acid; carbon; or a mixture thereof. The noble metal supported on the titanium silicate catalyst is preferably used. When the noble metal is supported on a carrier other than titanosilicate, it is possible that a carrier supporting the noble metal is mixed with the titanosilicate catalyst, and the mixture is used as the catalyst.

As a method for producing a noble metal catalyst, for example, a method of making a carrier support a noble metal, and then reducing it is known. For making the carrier support the noble metal compound, a conventionally known method such as impregnation may be used.

When a reducing gas is used in the reduction method, a reduction treatment in which a solid noble metal compound supported on a carrier is filled in an appropriate tube for filling, and a reducing gas is injected into the tube may be exemplified. Examples of the reducing gas include hydrogen, carbon monooxide, methane, ethane, propane, butane, ethylene, propylene, butene, butadiene, or mixed gases of two or more gases selected therefrom. Of these, hydrogen is preferable. The reducing gas may be diluted with a diluent gas such as nitrogen, helium, argon, steam, or a mixture of two or more kinds thereof.

In the noble metal catalyst, the noble metal is contained in a content of, for example, 0.01 to 20% by weight, preferably 0.1 to 5% by weight. The noble metal is used in an amount of, for example, 0.00001 part by weight or more, preferably 0.0001 part by weight or more, more preferably 0.001 part by weight or more per 1 part by weight of the titanosilicate catalyst. The noble metal is used in an amount of, for example, 100 parts by weight or less, preferably 20 parts by weight or less, more preferably 5 parts by weight or less per 1 part by weight of the titanosilicate catalyst.

Examples of the solvent used in the reaction step include water, organic solvents, and mixtures thereof.

Examples of the organic solvent include alcohol solvents, ketone solvents, nitrile solvents, ether solvents, aliphatic hydrocarbons, aromatic hydrocarbons, halogenated hydrocarbons, ester solvents, and mixtures thereof.

Examples of the alcohol solvent include aliphatic alcohols having 1 to 8 carbon atoms such as methanol, ethanol, isopropanol and t-butanol; and glycols having 2 to 8 carbon atoms such as ethylene glycol and propylene glycol. As a preferable alcohol solvent, for example, monohydric alcohols having 1 to 4 carbon atoms may be exemplified, and t-butanol is more preferable.

Examples of the aliphatic hydrocarbon include aliphatic hydrocarbons having 5 to 10 carbon atoms such as hexane and heptane. Examples of the aromatic hydrocarbon include aromatic hydrocarbons having 6 to 15 carbon atoms such as benzene, toluene and xylene.

Examples of the nitrile solvent include alkylnitriles having 2 to 4 carbon atoms such as acetonitrile, propionitrile, isobutyronitrile and butyronitrile, and benzonitrile. Acetonitrile is preferable.

As the solvent used in the reaction step, monohydric alcohols having 1 to 4 carbon atoms, acetonitrile, and the like are preferable in terms of the catalyst activity and the selectivity.

As acetonitrile, for example, crude acetonitrile, which is generated as a by-product in the production step of acrylonitrile, and purified acetonitrile can be used.

Examples of the impurity, that is, components other than acetonitrile, contained in crude acetonitrile include water, acetone, acrylonitrile, oxazole, allyl alcohol, propionitrile, hydrocyanic acid, ammonia, copper, and iron. Copper and iron are preferably contained in a trace amount of 1% by weight or less. Acetonitrile has a purity of, for example, 95% by weight or more, preferably 99% by weight or more, more preferably 99.9% by weight or more.

A mixed solvent of water and an organic solvent may be used as the solvent. In the mixed solvent, a preferable weight ratio of water and the organic solvent is, for example, from 0:100 to 50:50, preferably from 10:90 to 40:60.

The solvent is supplied in an amount of, for example, 0.02 to 70 parts by weight, preferably 0.2 to 20 parts by weight, more preferably 1 to 10 parts by weight per 1 part by weight of the olefin supplied.

The lower limit of the reaction temperature in the reaction step may be, for example 0° C., preferably 40° C. The upper limit of the reaction temperature in the reaction step may be, for example 200° C., preferably 150° C.

The lower limit of the reaction pressure (gauge pressure) in the reaction step may be a pressure of, for example 0.1 MPa, preferably 1 MPa, more preferably 20 MPa, further more preferably 10 MPa.

When hydrogen peroxide is generated from oxygen and hydrogen for use in the reaction step, it is preferable to continuously generate hydrogen peroxide by continuously supplying oxygen and hydrogen.

When hydrogen peroxide is generated from oxygen and hydrogen for use in the reaction step, the partial pressure ratio of oxygen to hydrogen, which are supplied into a reactor, may be, for example, oxygen:hydrogen=1:50 to 50:1, preferably oxygen:hydrogen=1:10 to 10:1. An oxygen partial pressure higher than oxygen:hydrogen=1:50 is preferable, because the production speed of an oxirane compound tends to increase, and an oxygen partial pressure lower than oxygen:hydrogen=50:1 is also preferable, because it tends to reduce the amount of by-products produced by reducing a carbon-carbon double bond of an olefin with a hydrogen atom, and to improve selectivity to an oxirane compound.

A mixed gas of oxygen and hydrogen is preferably handled in the presence of a diluent gas. Examples of the gas used for dilution include nitrogen, argon, carbon dioxide, methane, ethane, and propane. Nitrogen and propane are preferable, and nitrogen is more preferable.

As to the mixing ratio of oxygen, hydrogen, an olefin and a diluent gas, the case where they are used in the state of a mixture and in which propylene is used as an olefin and a nitrogen gas is used as a diluent gas will be explained. A mixing ratio in which the total concentration of hydrogen and propylene is 4.9% by volume or less and the oxygen concentration is 9% by volume or less, and a mixing ratio in which the total concentration of hydrogen and propylene is 50% by volume or more and the oxygen concentration is 50% by volume or less are preferable.

An oxygen gas and air containing oxygen may be used as oxygen. Examples of the oxygen gas include an oxygen gas produced by an inexpensive pressure swing method, and an oxygen gas having a high purity produced by cryogenic separation.

When hydrogen peroxide is continuously generated from oxygen and hydrogen for use in the reaction step, oxygen is supplied to a reactor in an amount of, for example, 0.005 to 10 moles, preferably from 0.05 to 5 moles per 1 mole of an olefin supplied to the reactor.

As hydrogen, for example, hydrogen obtained by steam-reforming of hydrocarbon can be used. Hydrogen has a purity of, for example, 80% by volume or more, preferably 90% volume or more.

When hydrogen peroxide is continuously generated from oxygen and hydrogen for use in the reaction step, hydrogen is supplied to a reactor in an amount of, for example, 0.005 to 10 moles, preferably from 0.05 to 5 moles per 1 mole of an olefin supplied to a reactor.

When hydrogen peroxide is continuously generated from oxygen and hydrogen for use in the reaction step, it is preferable that a buffer is put in a reactor, because there is a tendency that a decrease in the catalyst activity is prevented, the catalyst activity is further increased, and efficiency of utilization of oxygen and hydrogen is increased. Here, the buffer refers to a salt capable of providing buffering action to the hydrogen ion concentration of the reaction mixture in the reaction step (hereinafter may be referred to as a “reaction solution of the reaction step”).

It is preferable to dissolve the buffer in the reaction solution of the reaction step. When hydrogen peroxide is continuously generated from oxygen and hydrogen for use in the reaction step, the buffer may be previously contained in a noble metal complex. One of such methods is a method of making a carrier support an ammine complex such as Pd tetraamminechloride by an impregnation method or the like, and reducing the resulting product, whereby, while ammonium ions remain, a buffer is generated in a reaction solution of the reaction step. The buffer is added in an amount not exceeding the solubility of a buffer used in a solvent in the reaction step, preferably 0.001 mmol to 100 mmol per 1 kg of a solvent, for example.

Examples of the buffer include buffers containing 1) an anion selected from the group consisting of a sulfate ion, a hydrogen sulfate ion, a carbonate ion, a hydrogen carbonate ion, a phosphate ion, a hydrogen phosphate ion, a dihydrogen phosphate ion, a hydrogen pyrophosphate ion, a pyrophosphate ion, a halogen ion, a nitrate ion, a hydroxide ion and a C₁-C₁₀ calboxylate ion, and 2) a cation selected from the group consisting of an ammonium, a C₁-C₂₀ alkyl ammonium, a C₇-C₂₀ alkyl aryl ammonium, an alkali metal and an alkaline earth metal.

Examples of the carboxylate ion having 1 to 10 carbon atoms include a formate ion, an acetate ion, a propionate ion, a butyrate ion, a valerate ion, a caproate ion, a caprylate ion, a caprate ion, and a benzoate ion.

Examples of the alkyl ammonium include tetramethyl ammonium, tetraethyl ammonium, tetra-n-propyl ammonium, tetra-n-butyl ammonium, and cetyl trimethyl ammonium. Examples of the alkali metal cation and alkaline earth metal cation include a lithium cation, a sodium cation, a potassium cation, a rubidium cation, a cesium cation, a magnesium cation, a calcium cation, a strontium cation, and a barium cation.

Preferable examples of the buffer include ammonium salts of an inorganic acid, such as ammonium sulfate, ammonium hydrogen sulfate, ammonium carbonate, ammonium hydrogen carbonate, diammonium hydrogen phosphate, ammonium dihydrogen phosphate, ammonium phosphate, ammonium hydrogen pyrophosphate, ammonium pyrophosphate, ammonium chloride and ammonium nitrate; and ammonium salts of a carboxylic acid having 1 to 10 carbon atoms, such as ammonium acetate. Preferable ammonium salts are, for example, ammonium dihydrogen phosphate and diammonium hydrogen phosphate.

When hydrogen peroxide is continuously generated from oxygen and hydrogen for use, a quinoid compound may be added to a reaction solution of the reaction step. When the quinoid compound exists, selectivity to an oxirane compound tends to be further improved.

Examples of the quinoid compound include the compound represented by the formula (1);

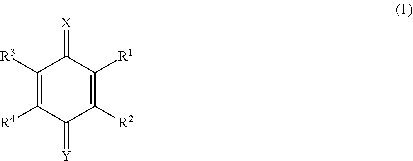

wherein R¹, R², R³ and R⁴ are each independently a hydrogen atom, or R¹ and R², or R³ and R⁴ are taken together with a carbon atom to which each of R¹, R², R³ and R⁴ is bonded to form a benzene ring which may have a substituent, or a naphthalene ring which may have a substituent; and X and Y are each independently an oxygen atom or an NH group).

Examples of the compound represented by the formula (1) include

1) a quinone compound (1A) in which R¹, R², R³ and R⁴ are each a hydrogen atom, and X and Y are each an oxygen atom in the formula (1);
2) a quinonimine compound (1B) in which R¹, R², R³ and R⁴ are each a hydrogen atom, X is an oxygen atom, and Y is an NH group in the formula (1); and
3) a quinondiimine compound (1C) in which R¹, R², R³ and R⁴ are each a hydrogen atom, and X and Y are each an NH group in the formula (1).

Other examples of the compound represented by the formula (1) include an anthraquinone compound represented by the formula (2):

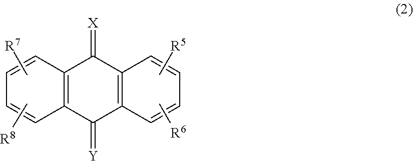

wherein X and Y are as defined in the formula (1); and R⁵, R⁶, R⁷ and R⁸ are each independently a hydrogen atom, a hydroxyl group or an alkyl group (for example, an alkyl group having 1 to 5 carbon atoms such as a methyl group, an ethyl group, a propyl group, a butyl group, or a pentyl group).

The compound represented by the formula (1) preferably has an oxygen atom as both of X and Y.

Examples of the compound represented by the formula (1) include quinone compounds such as benzoquinone and naphthoquinone; anthraquinones including 2-alkyl anthraquinone compounds such as 2-ethyl anthraquinone, 2-t-butyl anthraquinone, 2-amyl anthraquinone, 2-methyl anthraquinone, 2-butyl anthraquinone, 2-t-amyl anthraquinone, 2-isopropyl anthraquinone, 2-s-butyl anthraquinone and 2-s-amyl anthraquinone, polyalkyl anthraquinone compounds such as 1,3-diethyl anthraquinone, 2,3-dimethyl anthraquinone, 1,4-dimethyl anthraquinone, and 2,7-dimethyl anthraquinone, and polyhydroxyanthraquinone compounds such as 2,6-dihydroxyanthraquinone; p-quinoid compounds such as naphthoquinone and 1,4-phenanthraquinone; and o-quinoid compounds such as 1,2-phenanthraquinone, 3,4-phenanthraquinone and 9,10-phenanthraquinone.

Preferable examples of the compound represented by the formula (1) include anthraquinones, and 2-alkyl anthraquinone compounds (compounds of the formula (2) wherein X and Y are each an oxygen atom, R⁵ is an alkyl group, R⁶ is a hydrogen, and R⁷ and R⁸ are each a hydrogen atom).

The quinoid compound is used in the reaction step in an amount of, for example, 0.001 mmol to 500 mmol, preferably, for example, from 0.01 mmol to 50 mmol per 1 kg of a solvent contained in a reaction solution of the reaction step.

In the reaction step, it is possible to add a salt composed of an ammonium, an alkyl ammonium or an alkyl aryl ammonium to a reaction solution of the reaction step.

It is also possible to produce the quinoid compound by oxidizing a dihydro-form of a quinoid compound with oxygen or the like in a reaction solution of the reaction step. For example, hydroquinone or a dihydro-form of a quinoid compound such as 9,10-anthracene diol is added to a reaction solution of the reaction step, and hydroquinone or the dihydro-form is oxidized with oxygen in the reaction solution to generate a quinoid compound for use.

Examples of the dihydro-form of the quinoid compound include a dihydro-form of the compound represented by the formula (1), which is represented by the formula (3):

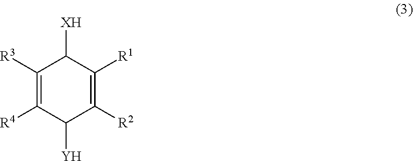

wherein R¹, R², R³, R⁴, X and Y are as defined above; and
a dihydro-form of the compound represented by the formula (2), which is represented by the formula (4):

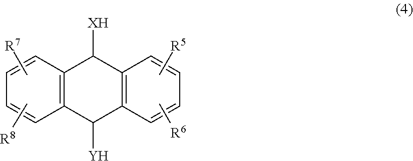

wherein X, Y, R⁵, R⁶, R⁷ and R⁸ are as defined above.

In the formula (3) and the formula (4), an oxygen atom is preferable as X and Y.

Preferable examples of the dihydro-form of the quinoid compound include dihydro-forms corresponding to the preferable quinoid compounds described above.

Procedures in the reaction step in the present invention may be continuously performed. Examples thereof include a step of continuously supplying hydrogen peroxide and an olefin into a reactor in which a solvent, a titanium silicate catalyst, and, if necessary, a buffer, a quinoid compound and the like are put, reacting them in the reactor, and continuously supplying the thus obtained reaction solution to a decomposition tank described later.

When hydrogen peroxide is generated from oxygen and hydrogen as described above, a noble metal catalyst is further put in the reactor, and while oxygen and hydrogen are continuously supplied into the reactor to continuously generate hydrogen peroxide in the reactor, a reaction solution containing hydrogen peroxide and an olefin oxide is continuously obtained. Oxygen, hydrogen and the olefin may also be continuously supplied in the state of a mixed gas, which contains a diluent gas, if necessary.

It is preferable that the reactor has a mixing means such as mixing blades. When the reactor has the mixing means, there is a tendency that hydrogen peroxide and the titanium silicate catalyst are efficiently mixed.

Examples of the concrete embodiment of the reactor include a reactor of reference number (3) shown in FIG. 1, (hereinafter may be referred to as a reactor (3)). That is, the reactor (3) has paddle blades in the inside. To the reactor (3) a tube (5) for continuously supplying a mixed gas of oxygen, hydrogen and an olefin to the reactor (3), and a tube (8) for continuously supplying the reaction solution from the reactor (3) to a decomposition tank (4) described later are attached. The reaction solution is continuously supplied from the reactor (3) to a tube (6).

The number of reactors used in the reaction step may be multiple. Concrete embodiments of the reactor include reactors of reference numbers (1) to (3) shown in FIG. 1 (which may be referred to as a reactor (1), a reactor (2) and a reactor (3), respectively).

That is, the reactor (1) has paddle blades in its inside. To the reactor (1) a tube (5) for continuously supplying a mixed gas containing oxygen, hydrogen and an olefin to the reactor (1), and a tube (6) for continuously supplying the reaction solution from the reactor (1) to the reactor (2) are attached. The reaction step is performed in the reactor (1), and a reaction solution obtained is continuously supplied to the reactor (2) through the tube (6) connected to the reactor (2).

The reactor (2) has paddle blades in its inside. To the reactor (2) a tube (5) for continuously supplying a mixed gas containing oxygen, hydrogen and an olefin to the reactor (2), and a tube (7) for continuously supplying a reaction solution from the reactor (1) to the reactor (2) are attached. The reaction step is performed in the reactor (2), and a reaction solution obtained is continuously supplied to the reactor (3) through the tube (7) connected to the reactor (3).

When the reaction solution is supplied from the reactor to a decomposition tank, it is preferable to supply the reaction solution from which the titanosilicate catalyst and the noble metal catalyst are removed to the decomposition tank. Specific examples thereof include a method of supplying a supernatant of the reaction solution containing almost no catalyst components used in the reactor, and a method of separating the catalyst components through a filter placed at a tube for continuously supplying the reaction solution from the reactor to the decomposition tank, or before or after the tube.

In the case of using multiple reactors, similarly to the above, when the reaction solution is supplied from one reactor to another reactor, it is preferable to supply a reaction solution from which a titanosilicate catalyst and a noble metal catalyst are removed to the different reactor.

The present invention further includes a step in which the reaction solution obtained in the reaction step is mixed with a reducing agent containing at least one selected from the group consisting of a sulfide and hydrazine (hereinafter may be referred to as a decomposition step).

The decomposition step is performed after the reaction step. The reaction solution used in the decomposition step may contain the olefin oxide produced. In the decomposition step in the present invention, even if decomposition is performed in the presence of an olefin oxide, hydrogen peroxide can be decomposed without substantially decomposing the olefin oxide. In addition, in the decomposition step in the present invention, oxygen is hardly generated.

Examples of the sulfide used in the decomposition step include salts of S²⁻ such as sodium sulfide, potassium sulfide, ammonium sulfide, sodium hydrogen sulfide and zinc sulfide, and sodium sulfide is preferable. The sulfide may be an anhydrous sulfide or a sulfide containing crystal water.

The sulfide is used in an amount of, for example, 0.01 to 10 moles, preferably, for example, 0.1 to 1 mole per 1 mole of hydrogen peroxide contained in the reaction solution obtained in the reaction step.

Hydrazine used in the decomposition step may be in any state such as an aqueous solution, a hydrate (hydrazine hydrate), a sulfate, a carbonate, a phosphate, or a hydrochloride.

Hydrazine is used in an amount of, for example, 0.01 to 20 moles, preferably, for example, 0.2 to 2 moles per 1 mole of hydrogen peroxide contained in the reaction solution obtained in the reaction step.

In the decomposition step, the reaction solution may be used as it is, or a solvent may be added to the reaction solution to dilute the reaction solution with the solvent. When the reaction solution is diluted as above, the amount of a reducing agent dissolved therein can be increased.

Examples of the solvent include the same solvents as listed in the reaction step, and preferably the solvent contained in the reaction solution of the reaction step is used as a solvent for dilution.

The amount of the solvent used depends on the amount of hydrogen peroxide contained in the reaction solution in the decomposition step, and it is, for example, from 1 to 1000000 parts by weight, preferably from 10 to 500000 parts by weight, more preferably from 100 to 10000 parts by weight per 1 part by weight of a reducing agent.

The lower limit of the reaction temperature in the decomposition step is, for example, 0° C., preferably 20° C. The upper limit of the reaction temperature of the decomposition step is, for example, 200° C., preferably 150° C.

The pressure (gauge pressure) in the decomposition step may be the same as the pressure in the reaction step, or after the reaction step, the pressure may be decreased, and the decomposition may be performed under an ordinary pressure or a reduced pressure. It is preferable to perform the decomposition under the same pressure as that in the reaction step.

In the present invention, procedures in the decomposition step may be continuously performed. Specific examples thereof include procedures in which the reaction solution of the reaction step, and a reducing agent containing at least one selected from the group consisting of a sulfide and hydrazine are continuously mixed in a decomposition tank, and a solution containing an olefin oxide is continuously obtained.

In order to decrease the content of hydrogen peroxide contained in the reaction solution, the retention time of the reaction solution in the decomposition tank is at least 0.1 hour, preferably from 0.5 to 5 hours.

A concrete embodiment of the reactor is, for example, a decomposition tank of reference number (4) shown in FIG. 1 (hereinafter may be referred to as a decomposition tank (4)). That is, the decomposition tank (4) has paddle blades in its inside. To the reactor (4) a tube (9) for continuously supplying a reducing agent, and a tube (8) for continuously supplying the reaction solution from the reactor (3) are attached. Hydrogen peroxide is decomposed in the decomposition tank (4), and a solution containing the olefin oxide and a decreased amount of hydrogen peroxide can be continuously obtained through a tube (10).

The noble metal catalyst and the titanium silicate catalyst are not contained in the decomposition tank. In the decomposition tank, hydrogen peroxide is decomposed, but the olefin oxide is hardly decomposed.

Examples of the reactor used in the reaction step and the decomposition tank used in the decomposition step include a flow-through fixed bed reactor and a flow-through slurry complete mixing apparatus.

When the flow-through slurry complete mixing apparatus is used in the reaction step, it is preferable that the titanosilicate catalyst and the noble metal catalyst are filtered through a filter placed in the reactor or outside the reactor, and the filtered products are supplied into the reactor again. Specific examples thereof include a method in which a part of the catalysts in the reactor are continuously or intermittently taken out of the reactor, the catalysts are subjected to a regeneration treatment, if necessary, and then the resulting catalysts are supplied to the reactor; and a method in which a part of the catalysts in the reactor are continuously or intermittently exhausted, and a new titanosilicate catalyst and a new noble metal catalyst are added to the reactor in amounts equal to the exhausted amounts of the catalysts.

When the flow-through fixed bed reactor is used as the reactor in the reaction step, for example, a method in which, a reactor containing a catalyst having lowered productivity of an olefin oxide is used for regenerating the catalyst, the catalyst is subjected to a regeneration treatment in the reactor, and the reaction and the regeneration are alternately repeated may be carried out. In this case, it is preferable to use a catalyst molded using a molding agent, or the like.

The product of the decomposition step is subjected to a separation treatment such as distillation, whereby an olefin oxide can be obtained. After the decomposition step, the product is separated through, for example, a gas-liquid separation tower, a solvent separation tower, a crude propylene oxide separation tower, a propane separation tower, or a solvent purification tower into crude propylene oxide, a gas component mainly containing hydrogen/oxygen/nitrogen, recovered propylene, a recovered solvent and a recovered quinone compound. It is preferable that the recovered propylene, the recovered solvent and the recovered quinone compound are supplied to the reaction step again for recycle. When the recovered propylene contains impurities such as propane, cyclopropane, methyl acetylene, propadiene, butadiene, butanes, butenes, ethylene, ethane, methane and hydrogen, it may be recycled through separation and purification, if necessary.

Hitherto, a mixture of unreacted propylene, hydrogen peroxide, and propylene oxide as a product, is supplied to a distillation zone; the resulting product is separated into an overhead fraction containing propylene, propylene oxide, and the like and a bottom fraction containing hydrogen peroxide, and the like; the bottom fraction is usually supplied to a decomposition zone in which a decomposition catalyst capable of decomposing hydrogen peroxide is held; and hydrogen peroxide is decomposed in the decomposition zone. According to the production method of the present invention, however, hydrogen peroxide contained in the mixture can be decomposed without distillation of the mixture.

### EXAMPLES

The present invention will be explained in more detail by means of examples below.

### Example 1

### Preparation of Titanosilicate Catalyst

In an autoclave, 112 g of TBOT (tetra-n-butyl orthotitanate), 565 g of boric acid, and 410 g of fumed silica (cab-o-sil M7D) were dissolved in 899 g of piperidine and 2402 g of pure water at room temperature (about 25° C.) under an air atmosphere by agitation, the mixture was stirred for further 1.5 hours, and the autoclave was sealed. Subsequently, the temperature in the autoclave was elevated over 8 hours while the solution in the autoclave was stirred, and the solution was kept at 160° C. for further 120 hours to obtain a suspended solution.

After the obtained suspended solution was filtered, the cake was washed with water until the pH of the filtrate became about 10. The obtained cake was dried at 50° C. to obtain a white powder containing water. To 15 g of the obtained powder was added 750 mL of 2 N nitric acid, and the mixture was heated for 20 hours under refluxing, and then the resulting product was filtered, washed with water until it was approximately neutral, and thoroughly dried at 50° C. to obtain 11 g of a white powder. An X-ray diffraction pattern of the white powder was measured by using an X-ray diffraction apparatus using copper K-α radiation; as a result, it was confirmed that the white powder was a Ti-MWW precursor. The powder was subjected to an IPC emission spectrometry and it was found that the powder contained titanium in a content of 1.65% by weight.

At room temperature, 2.28 g of the powder and about 80 ml of a solution of water/acetonitrile=20/80 (a weight ratio) containing 0.1% by weight of hydrogen peroxide were mixed, and the mixture was stirred for 1 hour and filtered to obtain a powder, which was used as a silicate catalyst.

### Example 1

### Reaction Step in Reactor Represented by Reference Number (1), and Supply of Hydrogen Peroxide

To an autoclave equipped with a jacket and having an internal volume of 300 ml were added 131 g of aqueous acetonitrile having a weight ratio of water/acetonitrile=30/70, and 2.28 g of the titanosilicate catalyst, and then the pressure in the autoclave was adjusted to an absolute pressure of 4 MPa with nitrogen and the temperature of the mixture in the autoclave was adjusted to 50° C. To the autoclave were continuously supplied 143 L (standard condition)/Hr of a nitrogen gas, 132 g/Hr of aqueous acetonitrile (the weight ratio of water/acetonitrile was 30/70) containing 0.7 mmol/kg of anthraquinone, 0.7 mmol/kg of ammonium dihydrogen phosphate and 3.1% by weight of hydrogen peroxide, and 36 g/Hr of liquid propylene. During the reaction, the reaction temperature was adjusted to 50° C., and the reaction pressure was adjusted to 4 MPa. After the pressure was returned to an ordinary pressure, while the titanosilicate catalyst was filtered through a sintered filter, gas-liquid separation was performed, and a liquid component and a gas component were continuously taken out. After 4 hours, the liquid component and the gas component were sampled at the same time, and each was analyzed by a gas chromatography to measure the content of propylene oxide contained in the liquid component or the gas component. The content of hydrogen peroxide contained in the liquid component was measured by titration using potassium permanganate.

Propylene oxide was produced in an amount of 100 mmol/hr. Hydrogen peroxide remained in a content of 1530 parts by weight per million in the liquid component.

### Example 2

### Reaction Step in Reactor Represented by Reference Number (1), and Generation of Hydrogen Peroxide

To an autoclave equipped with a jacket and having an internal volume of 300 ml were added 131 g of aqueous acetonitrile having a weight ratio of water/acetonitrile=30/70, 2.28 g of the titanosilicate catalyst, and 0.20 g of a catalyst in which 1% by weight of palladium is carried on activated carbon, and then the pressure in the autoclave was adjusted to an absolute pressure of 4 MPa with nitrogen, and the temperature of the mixture in the autoclave was adjusted to 50° C. To the autoclave were continuously supplied 146 L (standard condition)/Hr of a mixed gas having a composition of 3.6% by volume of hydrogen, 2.1% by volume of oxygen and 94.3% by volume of nitrogen, 90 g/Hr of aqueous acetonitrile (the weight ratio of water/acetonitrile was 30/70) containing 0.7 mmol/kg of anthraquinone and 3 mmol/kg of diammonium hydrogen phosphate, and 36 g/Hr of liquid propylene. During the reaction, the reaction temperature was adjusted to 50° C., and the reaction pressure was adjusted to 4 MPa. After the pressure was returned to an ordinary pressure, while the activated carbon catalyst and the titanosilicate catalyst were filtered through a sintered filter, gas-liquid separation was performed, and a liquid component and a gas component were continuously taken out. After 6 hours, the liquid component and the gas component were sampled at the same time, and each was analyzed by a gas chromatography to measure the content of propylene oxide contained in the liquid component or the gas component. The content of hydrogen peroxide contained in the liquid component was measured by titration using potassium permanganate.

Propylene oxide was produced in an amount of 50 mmol/hr. Hydrogen peroxide remained in a content of 760 parts by weight per million in the liquid component.

### Example 3

### Reaction Step in Reactor, Reaction Step in Reactor Represented by Reference Number (2) or (3), and Generation of Hydrogen Peroxide

To an autoclave equipped with a jacket and having an internal volume of 300 ml were added 131 g of aqueous acetonitrile having a weight ratio of water/acetonitrile=30/70, 2.28 g of the titanosilicate catalyst, and 0.198 g of a catalyst in which 1% by weight of palladium is supported on activated carbon, and then the pressure in the autoclave was adjusted to an absolute pressure of 4 MPa with nitrogen, and the temperature of the mixture in the autoclave was adjusted to 50° C. To the autoclave were continuously supplied 146 L (standard condition)/Hr of a mixed gas having a composition of 3.6% by volume of hydrogen, 2.1% by volume of oxygen and 94.3% by volume of nitrogen, 90 g/Hr of aqueous acetonitrile (the weight ratio of water/acetonitrile was 30/70) containing 0.7 mmol/kg of anthraquinone, 0.7 mmol/kg of ammonium dihydrogen phosphate and 10% by weight of propylene oxide, and 36 g/Hr of liquid propylene. During the reaction, the reaction temperature was adjusted to 50° C., and the reaction pressure was adjusted to 4 MPa. After the pressure was returned to an ordinary pressure, while the activated carbon catalyst and the titanosilicate catalyst were filtered through a sintered filter, gas-liquid separation was performed, and a liquid component and a gas component were continuously taken out. After 6 hours, the liquid component and the gas component were sampled at the same time, and each was analyzed by a gas chromatography to measure the content of propylene oxide contained in the liquid component or the gas component. The content of hydrogen peroxide contained in the liquid component was measured by titration using potassium permanganate.

Propylene oxide was produced in an amount of 36 mmol/hr. Hydrogen peroxide remained in a content of 980 parts by weight per million in the liquid component.

### Example 4

### Preparation of Reaction Solution Containing Hydrogen Peroxide and Propylene Oxide

As the reaction solution of the reaction step, 100 g of an aqueous acetonitrile solution (acetonitrile/water=7/3) containing 10% by weight of propylene oxide, 0.007% by weight of propylene glycol, 1414 ppm of hydrogen peroxide, 0.7 mmol/kg (in terms of an aqueous acetonitrile solution) of anthraquinone as the quinone compound, and 3 mmol/kg (in terms of an aqueous acetonitrile solution) of ammonium dihydrogen phosphate ((NH₄)₂HPO₄) as the buffer was prepared.

### (Decomposition Step: Decomposition Step in Decomposition Tank Represented by Reference Number (4))

After the temperature of the aqueous acetonitrile solution was adjusted to 70° C., 0.17 g (0.25 mole per 1 mole of hydrogen peroxide contained in the aqueous acetonitrile solution) of sodium sulfide nonahydrate (which may be referred to as Na₂S.9H₂O, or NAS) was added thereto, and the mixture was stirred at 70° C. Results of retention ratios of each of hydrogen peroxide (H₂O₂) and propylene oxide (PO), calculated assuming that the amount thereof just after mixing with sodium sulfide was 100, and the concentration of propylene glycol (PG), obtained by hydrolysis of propylene oxide, are summarized in Table 1 in time series. In addition, the results obtained in the case where the mixture was stirred without addition of sodium sulfide are also shown in Table 1.

As is apparent from Table 1, it is understood that the amount of H₂O₂ was decreased to 10% in 2 hours, but almost all of PO retained and the amount of PG was hardly increased.

TABLE 1

| | H₂O₂ retention | | PO retention | | PG concentration | |
|---|---|---|---|---|---|---|
| | ratio (%) | | ratio (%) | | (% by weight) | |
| Stirring | Mixed | | Mixed | | Mixed | |
| time | with | No | with | | with | |
| (minute) | NAS | NAS | NAS | No NAS | NAS | No NAS |
| 0 | 100 | 100 | 100 | 100 | 0.007 | 0.009 |
| 30 | 18 | 100 | 93 | 93 | 0.006 | 0.006 |
| 60 | 14 | 100 | 91 | 92 | 0.006 | 0.007 |
| 120 | 10 | 97 | 89 | 90 | 0.008 | 0.009 |
| 210 | 6 | 95 | 89 | 90 | 0.013 | 0.013 |

### Example 5

### Preparation of Reaction Solution Containing Hydrogen Peroxide and Propylene Oxide

As the reaction solution of a reaction step, 100 g of an aqueous acetonitrile solution (acetonitrile/water=7/3) containing 1273 ppm of hydrogen peroxide, 0.7 mmol/kg (in terms of an aqueous acetonitrile solution) of anthraquinone as the quinone compound, and 3 mmol/kg (in terms of an aqueous acetonitrile solution) of diammonium hydrogen phosphate ((NH₄)₂HPO₄) as the buffer was prepared.

### (Decomposition Step: Decomposition Step in Decomposition Tank Represented by Reference Number (4))

After the temperature of the aqueous acetonitrile solution was adjusted to 70° C., 0.073 g (0.50 mole per 1 mole of hydrogen peroxide contained in the aqueous acetonitrile solution) of hydrazine monohydrate (which may be referred to as NH₂NH₂.H₂O, or NN) was added thereto, and the mixture was stirred at 70° C. Results of retention ratios, calculated assuming that the amount thereof just after mixing hydrogen peroxide (H₂O₂) with hydrazine was 100, are summarized in Table 2 in time series.

TABLE 2

| Stirring time | H₂O₂ retention |
|---|---|
| (minute) | ratio (%) |
| 0 | 100 |
| 120 | 58 |
| 180 | 49 |
| 240 | 45 |

### INDUSTRIAL APPLICABILITY

According to the production method of the present invention, an olefin oxide having a decreased content of hydrogen peroxide can be provided without distillation for separating the olefin oxide from hydrogen peroxide.

### EXPLANATION OF REFERENCE NUMBERS

(1) to (3): reactor
(4): decomposition tank
(5): tube for supplying mixed gas containing oxygen, hydrogen, olefin and diluent gas
(6): tube for supplying reaction solution from reactor (1) to reactor (2)
(7): tube for supplying reaction solution from reactor (2) to reactor (3)
(8): tube for supplying reaction solution from reactor (3) to decomposition tank (4)
(9): tube for supplying reducing agent
(10): tube for obtaining solution containing olefin oxide

1. A method for producing an olefin oxide, comprising:
a reaction step of reacting hydrogen peroxide with an olefin in the presence of a solvent and a titanium silicate catalyst; and
a step of mixing a reducing agent containing at least one selected from the group consisting of a sulfide and hydrazine with the reaction solution obtained in the reaction step.

2. The method according to claim 1, wherein the reducing agent is sodium sulfide.

3. The method according to claim 1, wherein the reducing agent is a hydrazine hydrate or an aqueous solution of hydrazine.

4. The method according to claim 1, wherein the olefin is propylene, and the olefin oxide is propylene oxide.

5. The method according to claim 1, wherein the solvent is a mixed solvent of acetonitrile and water.

6. The method according to claim 1, wherein the titanium silicate catalyst is a Ti-MWW precursor having a molar ratio of silicon to nitrogen (an Si/N ratio) of 5 to 20.

7. A method for producing an olefin oxide, comprising:
a step of continuously adding hydrogen peroxide and an olefin to a reactor in which a solvent and a titanium silicate catalyst are contained, performing reaction in the reactor, and continuously supplying the obtained reaction solution to a decomposition tank; and
a step of continuously supplying the reaction solution obtained in the above-mentioned step, and a reducing agent containing at least one selected from the group consisting of a sulfide and hydrazine to a decomposition tank to continuously obtain a solution containing an olefin oxide.

8. A method for decreasing an amount of hydrogen peroxide in a solution containing an olefin oxide, comprising:
a step of mixing a solution containing hydrogen peroxide and an olefin oxide with a reducing agent containing at least one selected from the group consisting of a sulfide and hydrazine to decompose hydrogen peroxide.
